(19) United States
(12) Patent Application Publication
(10) Pub. No.: US 2006/0178568 A1
(43) Pub. Date: Aug. 10, 2006

(54) Rapid diagnostic assay

(76) Inventors: Dominick Danna, Syracuse, NY (US); Wendy Marie Scinta, Cazenovia, NY (US); Allan I. Krauter, Skaneateles, NY (US); Richard W. Newman, Auburn, NY (US); Andrew Jay Kugler, Albany, NY (US)

Correspondence Address: HISCOCK & BARCLAY, LLP, 2000 HSBC PLAZA, ROCHESTER, NY, 14604-2404, US

(21) Appl. No.: 11/267,647

(22) Filed: Nov. 4, 2005

Related U.S. Application Data
(63) Continuation-in-part of application No. 10/981,369, filed on Nov. 4, 2004.

(51) Int. Cl. A61B 5/00; C12Q 1/70; C12Q 1/68

(52) U.S. Cl. 600/300; 435/6; 435/5; 128/920

(57) ABSTRACT

Disclosed is a rapid and easy to use diagnostic tool that a point-of-care practitioner can use to specifically identify the cause of a disease, such as the upper respiratory infection (URI) pharyngitis. Such a disease has multiple potential causative pathogens and has a number of combined clinical manifestations. The diagnostic tool is rapid in order to provide the busy point-of-care practitioner with an assay result within a time that does not affect patient flow. The time usually available to such a practitioner is optimally less than 10 minutes, so that an assay that detects multiple pathogens rapidly is regarded as one that does so in less than 10 minutes. The diagnostic tool can be operated with minimal training and within the confines of said practitioner's environment. The diagnostic tool has specificity and sensitivity above those of the prior art devices. The tool is self-contained, which thereby helps to control the spread of infection and eases the burden of disposal of used equipment. The tool includes a diagnostic card configured to enable a plurality of nucleic acid diagnostic assays for rapidly detecting the presence or absence of multiple pathogens at the point-of-care. The tool includes a device that interacts with the card and that contains assay analysis means.

### CROSS-REFERENCE TO RELATED APPLICATION

This application is a continuation-in-part of copending U.S. patent application Ser. No. 10/981,369, filed Nov. 4, 2004.

### FIELD OF THE INVENTION

The present invention relates to medical diagnostics and, more specifically, relates to rapid nucleic acid diagnostics.

### BACKGROUND OF THE INVENTION

The ability to rapidly and to accurately diagnose medical conditions provides significant benefits to patients, care-practitioners, and the payers. The desire for a rapid turnaround time creates a need to facilitate testing that can be delivered at the point-of-care, which is the site where real time or near real time diagnostic testing can be done so that the resulting test is performed more efficiently than comparable tests that do not employ this system. Point-of-care testing is testing at or near the site of patient care, wherever that medical care is needed. A rapid turnaround time in less than 10 minutes for test results provides many benefits including real time evidence-based decisions, immediate treatment of patients, minimization of unnecessary tests, minimization of unnecessary empiric medications, and fewer patients lost to follow up. These benefits, when combined with diagnosis accuracy, provide significant cost efficiencies throughout the medical system.

The benefits of rapidly diagnosing medical conditions at the point-of-care have been recognized by others. For instance, in U.S. Pat. No. 6,394,952 there is disclosed a point-of-care diagnostic system that is designed to process patient data from numerous point-of-care diagnostic tests or assays, including immunoassays, electrocardiograms, X-rays and other tests, and to provide an indication of a medical condition or risk or absence thereof. The processing of numerous sets of patient data is intended to aid the point-of-care practitioner in diagnosing various types of medical conditions.

In the point-of-care practitioner's setting, there are a number of combined clinical manifestations caused by a disease group. Such disease groups include upper respiratory infections, lower respiratory infections, sexually transmitted diseases, and others. Although the present application focuses on upper respiratory infections (URI) as an example of a diagnostic group, one skilled in the art recognizes that the present invention has applicability to other broad diagnostic groups as well.

Cardiovascular applications also exist within the field of molecular biology for rapid infectious disease testing using nucleic acids. For example, infectious diseases have been shown to be responsible for valvular diseases (GABHS in rheumatic heart disease), and inflammation of the heart tissue itself (as in a viral pericarditis or myocarditis). A sample of the tissue or fluid surrounding the heart could be used to rapidly predict the causative agent leading to a rapid, accurate treatment plan. In addition, testing for specific alleles of genes could be used to predict those at risk of myocardial infarction. For instance, specific alleles of a gene have recently been identified that confer approximately twice the average risk of myocardial infarction in carriers.

Cancer detection and treatment can be enhanced by using nucleic acid testing for rapid detection of a specific chromosomal abnormality. For example, CML involves a single translocation of chromosomes 9 and 22, creating the Philadelphia chromosome. Application of a mutation-specific primer (such as those used by the Invader assay) can detect this abnormality and diagnosis and treatment can then occur promptly. Nucleic acid testing also can apply to the diagnosis of constitutional genetic disorders involving mutations, such as the point mutation of Factor V Leiden disorder. Factor V Leiden causes the blood to become hypercoaguable, predisposing one to the formation of blood clots. Rapid turnaround times for this disorder can impact and improve postsurgical care, and can be used before prescribing certain medications, such as estrogens or birth control pills.

There are many pathogens, viral and bacterial, that are responsible for a combination of clinical manifestations, such as swollen glands, fever, and sore throat. These clinical manifestations are associated with pharyngitis, an upper respiratory infection. Many viruses that cause pharyngitis are not affected by available treatments. Other causes of pharyngitis, which could be responsible for long-term complications, are treatable and the diagnosis of these pathogens is very important. These include the bacterium Streptococcus Pyogenes, and the viruses Influenza A, Influenza B, and Epstein-Barr Virus (EBV). There is a strong possibility that there will be treatments developed for other causes of pharyngitis and, when this occurs, these pathogens can be added to the invention as herein described.

Each year in the United States, there are over 72 million office visits due to upper respiratory infections. Patients who present with the symptoms of a fever, sore throat, and swollen glands may be infected with Streptococcus Pyogenes, Influenza A, Influenza B, Epstein-Barr Virus (EBV), or a variety of less serious pathogens. The diagnosis is complicated by imprecise clinical signs and symptoms and by inaccuracies of current testing strategies. As discussed, the large majority of infectious agents responsible for pharyngitis are viruses. Only 5 to 15 percent of adult cases are caused by bacteria, with Group A beta hemolytic streptococcus (GABHS) being the most common etiology. In children, GABHS is far more prevalent accounting for approximately 30 percent of pharyngitis cases. Respiratory illness caused by influenza is difficult to distinguish from illness caused by other respiratory pathogens based on symptoms alone.

Despite the preponderance of viral causative agents, 76% of adults and 71% of children diagnosed with pharyngitis in 1992 were treated with antibiotics. The high rate of use of antibiotics is concerning because of the issue of drug resistance and the high cost of antibiotics. In recent years, there has been an increased awareness of the overuse of antibiotics both in the medical community and the public at large. An accurate and rapid diagnostic tool that is available to a point-of-care practitioner to help distinguish between viral, bacterial, fungal, and parasitic infections would greatly reduce the high rate of use of antibiotics because the point-of-care medical practitioner would have an accurate diagnosis and subsequent treatment plan completed before the patient left the office.

There are current diagnostic tests that are available for pharyngitis and other upper respiratory infections, tests such as culture, serology, immunofluorescence assays, rapid antigen testing, and laboratory-based Polymerase Chain Reaction (PCR) assay testing to name a few. Each of these is performed using different methodology and devices.

There are many practice patterns used by physicians when a patient presents with symptoms of pharyngitis. For example, some practitioners run a rapid strep antigen test. However, due to variable accuracy of the test, many practitioners follow up a negative test result with a culture, prescribe antibiotics even after the negative test result, or do not use rapid tests. When a culture is used, one must either wait a day or more for the result before prescribing antibiotics, or start the course of antibiotic treatment immediately.

After the rapid strep antigen test, practitioners may then follow up with a rapid influenza test. If influenza is not diagnosed in the first 24-48 hours, treatment with antivirals is not effective. The sequential nature of current pharyngitis diagnostic practices also leads to additional cost due to testing and follow-up office visits, particularly in the case of mononucleosis, which tends to be a diagnosis of exclusion. This serial testing technique is labor intensive and inefficient.

The present invention utilizes nucleic acid testing to differentiate the treatable and non-treatable causes of pharyngitis. Of course, nucleic acid based assays have been known in the art for some time. The invention of PCR ushered in a new era in the biological sciences and is described in U.S. Pat. Nos. 4,683,195 and 4,683,202. Nucleic acid testing offers some significant advantages over other testing methods such as immunoassays. Nucleic acid testing is generally more accurate than antibody/antigen testing. Heretofore nucleic acid testing has been limited to a clinical laboratory setting using skilled technicians in a controlled environment. Nucleic acid testing is extremely beneficial to immunocompromised individuals, such as those on chemotherapy or with HIV. Such individuals cannot mount an immune response sufficient to produce a positive result on current rapid immunoassay tests. Another advantage of nucleic acid testing is that the sensitivity of nucleic acid testing allows for a single sample having a smaller volume than the sample needed to conduct immunoassays, or the single sample can be collected from one site such as the throat, which may contain the particular pathogen in smaller concentrations than other sample sites such as the nasal passage. An additional advantage to nucleic acid testing in the present invention is that this approach allows for the detection of a specific strain of a pathogen, such as influenza, so that if a pandemic event does occur, the medical community will be better prepared and limit the loss of life by providing additional time for vaccine development.

Nucleic acid PCR based-assays are typically performed on a large-scale basis in a clinical laboratory setting, although some have been contemplated on a fluid card. For instance, U.S. Pat. No. 5,994,056 addresses homogenous methods for nucleic acid amplification and detection. However, the inventions disclosed therein are only applicable to the laboratory setting using large automated equipment that typically includes 48-well or 96-well instruments. U.S. Pat. No. 6,440,725 describes an integrated fluid manipulation card that allows increased sensitivity in the detection of low-copy concentrations of analytes, such as nucleic acid. However, the device disclosed therein tests for only one pathogen per card and is not designed for rapid diagnosis in a time frame that is acceptable to point-of-care practitioners.

In addition, many of the aforementioned devices and methods for diagnosis are complicated and difficult to use. These devices must be used by trained technicians and can be prone to error if not conducted under strict guidelines. It would be preferable to supply a diagnostic device that is easy to use for even non-trained technicians. For instance, in the United States the Clinical Laboratory Improvement Amendments of 1988 (CLIA) established quality standard for all laboratory testing to ensure accuracy, reliability and timeliness of patient test results regardless of where the test is performed. Under CLIA, many federal requirements of the CLIA laws are waived if the test in question is determined by the Centers for Disease Control or by the Food and Drug Administration to be so simple that there is little risk of error. For example, some testing methods for glucose and cholesterol are waived along with some pregnancy tests, fecal occult blood tests, some urine tests, etc.

Therefore, there remains a need for a rapid and easy to use CLIA-waivable diagnostic tool that a point-of-care practitioner can use to specifically identify the cause of a disease, such as the URI pharyngitis that has common clinical manifestations (symptoms), and that has multiple potential causative pathogens. The diagnostic tool must be rapid in order to provide the busy practitioner with an assay result within a time that does not affect patient flow. The time usually available to a point-of care practitioner is optimally less than 10 minutes, so that an assay that detects multiple pathogens rapidly is regarded as one that does so in less than 10 minutes. The diagnostic tool must be easy to use so that the practitioner can operate the tool with minimal training and within the confines of the practitioner's environment. Preferably, the diagnostic tool must have specificity and sensitivity above those of the prior art devices. The tool is preferably self-contained, which thereby helps to control the spread of infection and eases the burden of disposal of used equipment.

### OBJECTS AND SUMMARY OF THE INVENTION

It is therefore an object of the present invention to provide a rapid and easy to use diagnostic tool that a point-of-care practitioner can use to specifically identify the cause of a clinical symptom having multiple potential causative pathogens.

It is another object of the present invention to provide a diagnostic tool that gives the point-of-care practitioner an assay result within a time that does not affect patient flow.

It is yet another object of the present invention to provide a diagnostic tool that gives the point-of-care practitioner an assay result in under 10 minutes.

Another object of the present invention is to provide a diagnostic tool that the point-of-care practitioner can operate with minimal training and within the confines of a typically busy point-of-care practitioner's environment.

It is an object of the invention to provide a diagnostic tool that is rapid and easy to use, that a point-of-care practitioner can use to specifically identify the cause of a clinical symptom having multiple potential causative pathogens, and that improves specificity and sensitivity over prior art devices.

It is an object of the invention to provide a single-use diagnostic tool that is rapid and easy to use, that a point-of-care practitioner can use to specifically identify the cause of a clinical symptom having multiple potential causative pathogens, and that is self-contained thereby helping to control the spread of infection and ease the burden of disposal of used equipment.

It is yet another object of the present invention to provide a diagnostic tool that is rapid and easy to use and that a point-of-care practitioner can use to specifically identify the cause of a clinical symptom having multiple potential causative pathogens while only requiring the practitioner to obtain a single sample from the patient.

It is still yet another object of the present invention to provide a diagnostic tool that is rapid and easy to use, and that a point-of-care practitioner can use to specifically identify the cause of a clinical symptom having multiple potential causative pathogens while only requiring the practitioner to obtain a single sample from a single site from the patient.

These and other objects are met by providing a diagnostic tool that utilizes nucleic acid testing and that allows the point-of-care practitioner to test for multiple types or categories of pathogens using one procedure involving a single specimen sample and a single card. A nucleic acid approach on a single card allows the point-of-care practitioner to diagnose the cause of a common clinical manifestation or symptom using only one testing card regardless of what pathogen is the underlying cause, be it bacterial, viral, fungal, parasitic or a combination thereof.

### BRIEF DESCRIPTION OF THE DRAWINGS

FIG. 1 is a view of the system embodying the present invention.

FIG. 2a is a view of a sample collection device that is a part of the system embodying the present invention.

FIG. 2b is a plan view of an alternative embodiment of a sample collection device that is a part of the system embodying the present invention.

FIG. 3 is a schematic view of a microfluidic card that embodies the present invention.

FIG. 4a is a cross-sectional view of the sample insertion chamber of the microfluidic card embodying the present invention.

FIG. 4b is an exploded plan view of an alternative embodiment of the support mechanism and actuator rod used in the sample insertion chamber of the microfluidic card of the present invention.

FIG. 5 is a top plan view of the desktop device which is a part of the system of the present invention.

FIG. 6 is a schematic view of an alternative embodiment of a desktop device and microfluidic card of the present invention.

FIG. 7 is a schematic view of the network and process that is enabled by the rapid diagnostic card of the present invention.

### DETAILED DESCRIPTION

The invention herein described provides a diagnostic test that can be performed rapidly and at the point-of-care, such as in a doctor's office, at a bedside, in the field, or in an emergency room. As used herein, point-of-care testing refers to real time or near real time diagnostics that can be done in a rapid time frame so that the resulting test is performed faster than comparable tests that do not employ this system. Point-of-care testing is testing at or near the site of patient care, wherever that medical care is needed.

As used herein, diagnosis refers to a predictive process in which the presence, absence, severity or course of treatment of a disease, disorder or other medical condition is assessed. As used herein, a patient or subject includes any mammals for which diagnosis is contemplated. Humans are the preferred subjects.

The present invention is directed to detecting selected nucleic acids from a sample. The nucleic acid in the sample will be a sequence of genomic DNA and/or other nucleic acids, such as mitochondrial DNA, messenger RNA, ribosomal RNA, or viral RNA. Suitable nucleic acid samples include single or double-stranded DNA or RNA. Each of the selected nucleic acids is specific to one of the pathogens that is being detected. The detection of messenger RNA gives the ability to differentiate between live and dead pathogens. Messenger RNA is a reflection of active replication and typically degrades in approximately 30 minutes, so the detection of messenger RNA is a good indicator of an active pathogen.

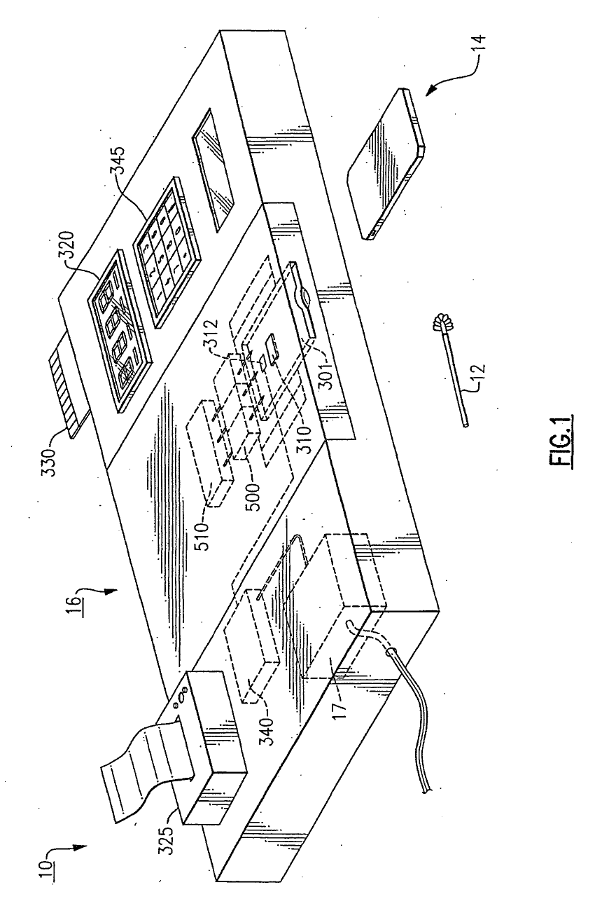

Referring now to FIG. 1, the diagnostic tool 10 described herein uses a sample collection device 12 that interacts with a self-contained card 14, which is designed for the point-of-care practitioner to use in the specific diagnosis of an upper respiratory infection and which represents one embodiment of the present invention. The card 14 is exposed to the sample and then is placed in mechanical interaction with a portable and/or desktop device 16, and is preferably in fluid communication with the device 16 as will be discussed in more detail below. The device 16 is powered through a power supply 17 as is well known in the art. As mentioned herein above, the specific diagnosis of a number of broad clinical groups can utilize the present invention, including but not limited to, upper respiratory infections, sexually transmitted diseases, and uro-genital conditions. One could select other groups of different pathogens to meet other broad clinical manifestations or be adapted to diagnose common clinical manifestations in specific environments such as the tropics or a battlefield environment. We herein describe a diagnostic tool that rapidly and efficiently tests for multiple pathogens on a single card, the pathogens being selected for their common clinical manifestations.

Use of the present diagnostic tool includes initially collecting a sample from the patient. There are known in the art various methods of collecting samples. For example, in the diagnosis of the specific cause of pharyngitis, a sample is typically collected from the throat, mouth or nose of the patient by using a cotton swab located at the distal end of a shaft. Those skilled in the art would recognize that there are various methods of collecting samples and the method that is chosen is somewhat dependent upon the particular sample that is desired.

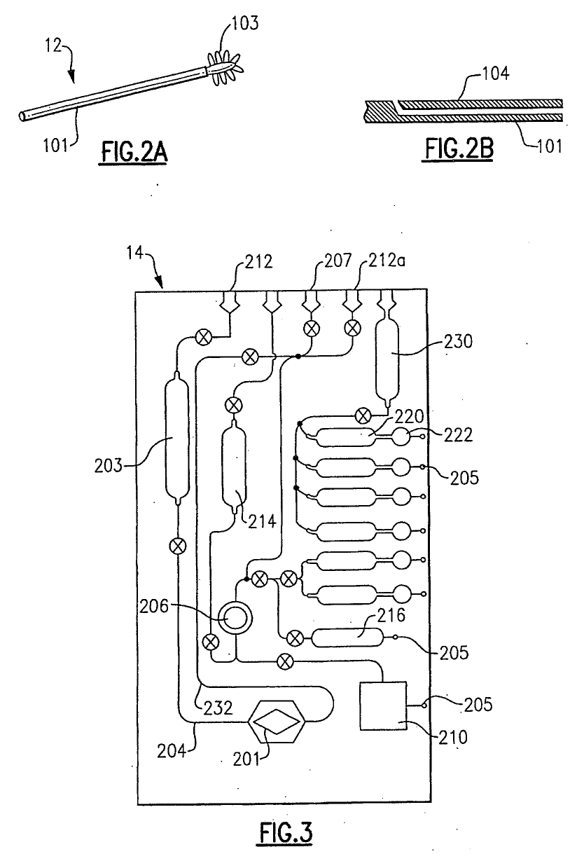

In the preferred embodiment, the sample collection device collects a targeted amount of sample. Of course, there is an advantage in knowing the precise amount of sample that is collected because certain assays require a requisite amount of sample fluid in order to give accurate results. In some situations it will be preferable to limit the amount of sample introduced into the card 14 in order to minimize the amount of waste material that will be produced. The sample size can be limited by the configuration of the sample collection device or by the card, which can employ configurations in the size of the acquisition port or solid-state support that will be referred to in more detail below. In addition, by knowing the amount of sample introduced into the card, one skilled in the art would recognize methods to quantify the amount of pathogen present in the sample. Referring now to FIG. 2a, there is shown one example of a sample collection device 12, or swab. The swab 12 includes a shaft 101 which is of a suitable length to allow the care practitioner to grasp the shaft 101 at the proximal end and collect a sample from the back of the throat of the patient. At the distal end of the shaft 101 there is located a single or plurality of bristles 103. The bristles can be manufactured from any material that creates a surface tension with the targeted sample fluid, for instance a hydrophilic plastic. The bristles 103 have a predetermined amount of surface area that creates surface tension between the bristles 103 and the target sample fluid, resulting in a specifically selected amount of sample fluid being retained on the swab 12.

In an alternative embodiment as shown in FIG. 2b, the swab 12 has a capillary tube 104 located at the distal end of the shaft 101 rather than the bristles described above. The capillary tube 104 acquires a liquid sample by coming into contact, say with fluid at the back of the throat, wherein capillary action draws a selected amount of sample into the tube 104. The capillary tube 104 can include solid phase material such as, but not limited to, a glass-mesh Filter, in order to hold the sample during subsequent steps of the diagnostic procedure. Additionally, and as an alternative embodiment, the same solid phase material that is used to collect the sample can be used as a solid support in the card 14 for the lysing, washing, and other assay steps that are further described herein below.

Once the sample has been obtained it must be deposited into the card 14. Referring now to FIG. 3, there is shown a preferred embodiment of the card 14, The card 14 is designed to initially accept the sample fluid and then separate analytes, specifically nucleic acids, from the fluid sample. The desired analytes comprise nucleic acids from multiple groups of pathogens, including viruses, bacteria, parasites, and/or fungi. As used herein, the term “nucleic acid” refers to any synthetic or naturally occurring nucleic acids, such as DNA or RNA, in any possible configuration; i.e., in the form of double-stranded nucleic acid, single-stranded nucleic acid, or any combination thereof.

The card 14 has formed therein an acquisition port 201 for introducing the sample into the card 14. The sample is deposited on a solid support structure (not shown), which is located in the acquisition port 201. Those skilled in the art would recognize various materials that are suitable for solid supports including, but not limited to, filters, beads, fibers, membranes, glass wool, filter paper, polymers, gels, and micro/nanostructures. The preferred embodiment includes a glass fiber substrate. The distal portion of the swab 12 containing the sample is introduced into the card 14 via the acquisition port 201 and the swab 12 comes into contact or very close proximity to the solid support structure so that the sample is transferred to the support structure. The swab 12 is withdrawn from the acquisition port 201 and the acquisition port 201 is then sealed. There are known various methods of sealing a micro-fluidic card. For instance, a pressure sensitive adhesive can be applied to a flap of fluid impermeable material that could be used to cover and seal the acquisition port.

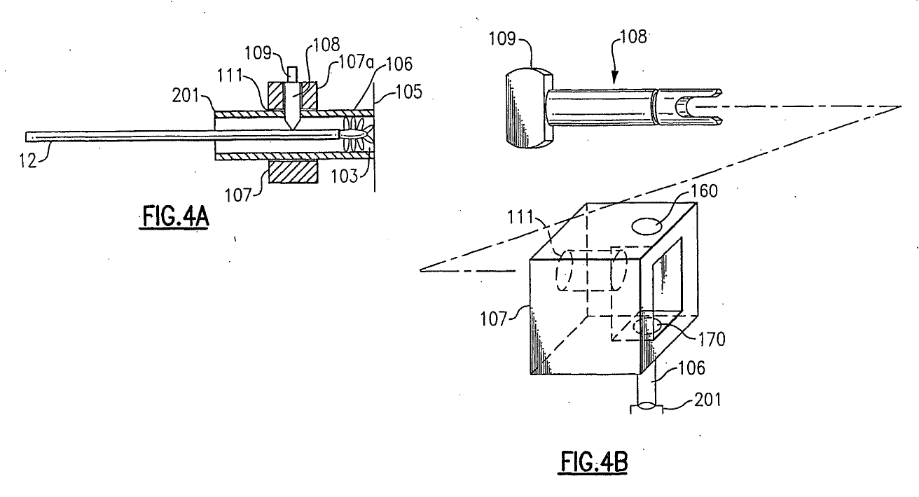

In an alternative arrangement the support structure could be used as the means for collecting the sample wherein the support structure is integral to the distal portion of the swab 12. Referring now to FIG. 4a, the distal portion of the swab 12 is inserted into acquisition port 201 (shown as tubular in FIG. 4a but depicted as flat in FIG. 3) of the card 14 after the swab has been used to obtain the target sample. The swab 12 is inserted until the sample-containing portion 103 of the swab 12 is substantially abutting the tip stop 105. The acquisition port 201 includes short tube 106 that is contained within the acquisition port 201. There is a support block 107, 107a that has a mechanical severing device 108, which is actuated at the end 109. Motion of the end 109 translates the severing device 108 through an opening 111 formed in the support block 107a across the diameter of the short tube 106 in order to cleanly break or sever the swab 12. After the swab 12 is severed, the proximal portion of the swab 12 is removed from the acquisition port 201. The severing device 108 is moved back to its original position. Finally, the portion of the acquisition port 201 in the remaining short tube 106 is squeezed against the support block 107 by the support block 107a, thereby effectively sealing the cartridge.

Referring now to FIG. 4b, as an alternative embodiment the portion of the acquisition port 201 that lies outside of the short tube 106 can be bent, also resulting in breaking the swab and sealing the cartridge. In this embodiment, the swab is inserted vertically down through hole 160, through tube 106, and into the acquisition port 201. The handle 109 is then rotated approximately 180 degrees in either direction, which bends the portion of the acquisition port 201 that lies outside of the short tube 106. This motion breaks the swab and squeezes the acquisition port 201 between the device 108 and the support block 107, thereby effectively sealing the card.

Referring now to FIGS. 1, 3 and 5, after the sample has been deposited onto the solid support structure, the card 14 is inserted into a portable or desktop device 16. The device 16 includes a slotted entry port 301 that aligns the card 14 so that the card is in position to interact with various components of the desktop device 16 as will be described in more detail below.

In order to amplify a target nucleic acid sequence in a sample, the sequence must be accessible to the components of the amplification system. In general, this accessibility is ensured by isolating the nucleic acids from the crude biological sample, the first step of which is to lyse the cells to provide access to the nucleic acids. A variety of techniques for extracting nucleic acids from biological samples are known in the art. For example, see those described in Maniatis et al., Molecular Cloning: A Laboratory Manual (New York, Cold Spring Harbor Laboratory, 1982); Arrand, Preparation of Nucleic Acid Probes, in pp. 18-30, Nucleic Acid Hybridization: A Practical Approach (ed Hames and Higgins, IRL Press, 1985); or, in PCR Protocols, Chapters 18-20 (Innis et al., ed., Academic Press, 1990). The preferred embodiment in the present invention is to chemically lyse the pathogens contained in the sample. One skilled in the art recognizes that there are numerous lysing fluids that can be utilized including many commercially available enzymes and detergents like TWEEN 80 or Triton X-100.

There is a lysis fluid stored in a reservoir 203 contained on the card 14. The lysis fluid is directed to the solid support contained in the acquisition port 201 through a fluid channel 204 formed in the card 14. The lysis fluid is directed to the acquisition port 201 by a pumping action that could be supplied in various ways, such as by an air supply port 212 supplied with positive air pressure from the desktop device 16 as will be described in more detail below. The excess air that accumulates in the card 14 is vented through air vents 205 located at selected positions on the card 14. The vents 205 are preferably filter vents as known in the art and allow for gas to pass through but contain liquids within the card 14.

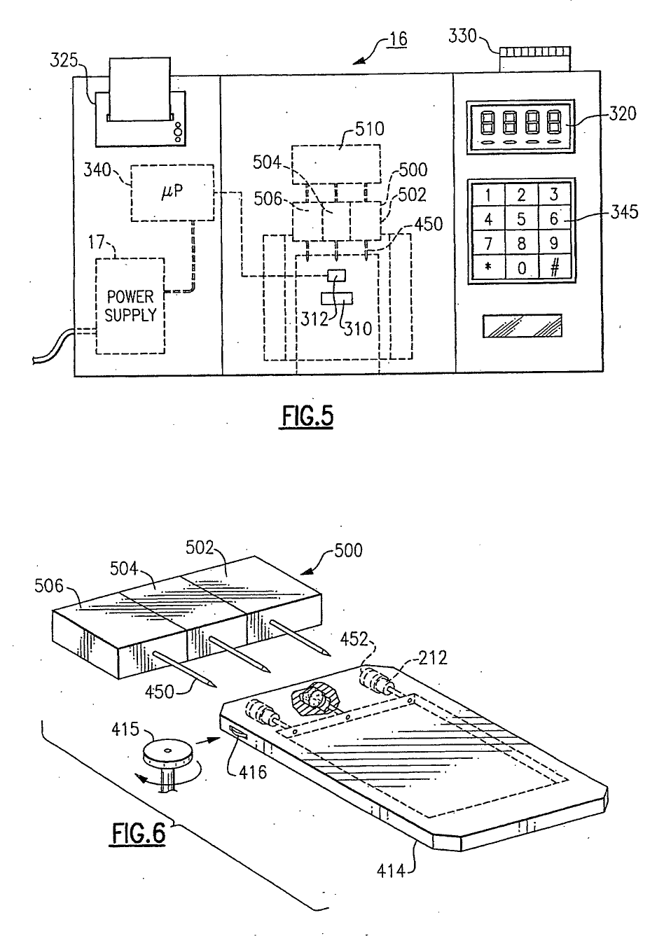

In an alternative embodiment depicted in FIGS. 5 and 6, the pumping action is supplied by a device 16 which provides mechanical energy to a microfluidic card 414 in order to power a peristaltic pump 416. The peristaltic pump 416 located in the card is driven by a mechanical drive 415 that is located on the desktop device 16. Indeed, one skilled in the art recognizes numerous ways of providing mechanical pumping action to a microfluidic card. In U.S. Pat. No. 6,743,399 to Weigl et al., there are disclosed numerous methods of propelling fluids through a microfluidic device. The methods disclosed in the patent include microfluidic cards that contain a power source internal to the structure for propelling the fluid through the device.

The lysis fluid flows over the solid support located in the acquisition port 201 and lyses the cells that are contained in the sample. The lysis fluid then flows through a channel 232 and over a nucleic acid capture filter 206 and subsequently into a waste compartment 210. The target nucleic acid from the lysed cells binds to the nucleic acid capture filter 206. One skilled in the art would recognize that several suitable materials could be used to form the nucleic acid capture filter 206.

Next, a wash solution, preferably ethanol, can be stored in a wash storage compartment on-board the card (not shown) or it can be stored in a reservoir on the device, as will be explained in more detail below. The ethanol is directed over the capture filter 206 via a channel 207 in order to remove any cellular debris that may have accumulated on the filter. The spent ethanol and cellular debris then flow to the waste compartment 210. Next, air is forced through air port 212a and over the capture filter 206 in order to dry the filter 206. An elution solution, many of which are commercially available, is stored in an elution fluid chamber 214. The elution fluid is pumped from the chamber 214 over the capture filter 206 and the target nucleic acid is released from the capture filter 206 and flows into the mix chamber 216. In the preferred embodiment, the elution solution flows back and forth over the capture filter 206 by alternately applying air pressure and vacuum at air port 212a in order to ensure that all nucleic acids that are released from the filter 206.

The elution solution now containing the target nucleic acid is directed to amplification tests wells 220. In the preferred embodiment, there are twelve separate amplification wells 220, which represent tests for four targeted pathogens. There is one amplification well for each of the four targeted pathogens and each of these wells receives one quarter of the elution solution. In addition, there are positive control wells and negative control wells for each pathogen, these wells being preloaded with the appropriate materials. The control wells are rehydrated with a buffered water solution that is stored either on-board the card 14 in a buffered water compartment 230 or the device 16. For ease of description, the figures contained herein depict only 6 amplification wells, which represent tests for only two targeted pathogens. One skilled in the art recognizes that the number of amplification wells 220 is determined by the number of targeted pathogens and the description herein is not meant to limit the configuration of the card 14.

At this point, the card carries out a polymerase chain reaction (PCR) amplification in each of the amplification wells 220. Those skilled in the art will recognize that the PCR process can be carried out as an automated process using a set of specifically selected reagents for each pathogen. In this process, the elution solution in each of the non-control amplification wells 220 is combined with an appropriate reaction mixture and these mixtures are then cycled through a denaturing temperature range, a primer annealing temperature range, and an extension temperature range. There are known in the art a number of ways to rapidly thermal cycle biological samples as is disclosed in U.S. Pat. No. 6,787,338 to Wittwer et al. Additional methods of performing rapid thermocycling are disclosed in U.S. Pat. No. 6,210,882 to Landers et al., which is hereby incorporated by reference in its entirety. By carefully controlling the speed and precise amplitude of the thermal cycling reaction, an acceptable amount of nucleic acid will be produced via the PCR. The reaction mixtures are subjected to approximately 35 thermal cycles in approximately 7 minutes. In the preferred embodiment, the thermal cycling, both heating and cooling phases, is produced by a Peltier device 310 located in a selected position in the desktop device 16 so that the Peltier device 310 interacts with the amplification wells 220. The Peltier device 310 is controlled by a microprocessor 340 in order to precisely control the duration and intensity of both the heating and cooling phases of the thermal cycles.

At this point, the reaction mixtures are transferred to detection wells 222 that contain a reagent that interacts with the target nucleic acid in a fashion that is easily detectable. In the preferred embodiment, the detection wells 222 contain SYBRGreen®, which provides a fluorescent signal if it attaches to the target nucleic acid and if it is properly illuminated. One skilled in the art recognizes that there are other suitable detection methods including, but not limited, to molecular beacons.

Referring to FIGS. 1, 3 and 5, any signal that is produced in the card detection wells 222 is detected by a fluorometer 312 that is housed in the desktop device 16. When the card 14 is seated in the device 16 in a proper configuration, the fluorometer 312 is positioned to read any signal generated in the detection wells 222. The fluorescent signal is analyzed with the microprocessor 340 by comparing the signal to the signals generated by the positive controls and negative controls. Results of the analysis are provided in a display window 320 or can be printed using a printing device 325 that can be integral to the device 16. Information regarding the results can also be transmitted to medical records/billings using a communications port 330, which is a two-way data transport system using a modem or wireless communications protocol. Additional information or instructions can be entered into the device via a keypad 345 or a wireless communications device as is known in the art.

The card 14 preferably includes a means to hold information, such as a bar code (not shown). One skilled in the art recognizes other ways to include information on card. The bar code contains information including, but not limited to, the type of card being inserted into the device, patient information, expiration dates, etc. The device 16 includes a means to read the information from the card 14. The interaction between the device 16 and the card 14 facilitates the rapid and easy transfer of information. As an example, the device 16 may be configured for one type of card (uro-genital testing), while the card 14 in use is actually an upper respiratory card. In this case, the device 16 determines the nature of the card 14 that is interacting with the device and then applies the correct configuration of the device (selection of reagents, thermal cycle times, etc.) for the particular card that has been inserted. Other uses for the information can include, for instance, an error detection function. For instance, the device 16 can generate an indicator signal to the practitioner for the need of a change in configuration of the device 16, or that the card has passed an expiration date.

Referring now to FIG. 5, the device 16, rather than the card 14, can house some of the components/reagents that are used in the diagnostic system. Referring to FIG. 3, it has been described above that the air pressure can be supplied to the card 14 through an air port 212 and 212a, such as shown, The air port 212 and 212a are placed into fluid communication with the desktop device when the card 14 is correctly seated in the desktop device. The desktop device can include one or more fluid communications means to supply air and/or other reagents to the card and includes a mechanical pump 510. For instance, referring to FIG. 6, any of the reagents could be stored on board the desktop device 16 in a single storage compartment or in multiple storage compartments/reservoirs 502, 504, 506. The reagents are then supplied to the card 414 through dedicated needles 450. The needles 450 pass through elastomeric seals 452 contained on the card 414 and the proper reagent reservoir is placed in fluid communication with the proper micro-fluidic channel on the card 414.

If a multiple number of reservoirs are employed, the reservoirs could be housed together in a reagent module 500 that is replaceable within the device 16. Different modules 500 could utilize specific reagents that are matched to the type of card that is being analyzed. As described above, one type of card might contain an upper respiratory panel for pharyngitis and another type of card would be used for uro-genital conditions, and the two cards might use different reagents because each card would be designed to detect different pathogens. The card will preferably include information storage means such as a bar code (not shown) that can be read by the device in order to assure that the proper reagent module 500 is in place in the desktop device. Of course, the information storage means could include many additional types of information that could be read by the device including, but not limited to, process variables, expiration dates, lot numbers, and patient information.

The module 500 can include several needles 450 that are in fluid communication with the appropriate reservoirs 502, 504, 506. The card 414 includes elastomeric seals 452 that are configured to accept the appropriate needle 450. When the card 414 is correctly inserted into the desktop device 16, the needles 450 extend through the elastomeric seals 452 and provide fluid communication between the appropriate reservoirs and the appropriate fluid channels on the card.

In use, a patient presents to a point-of-care practitioner with common clinical manifestations of a disease from a broad diagnostic group such as upper respiratory infections. One such disease is pharyngitis. For instance, the patient presents with a sore throat, swollen lymph nodes, and a fever. At an early point in the visit, the practitioner obtains a sample using a swab 12 from a single site, in this case either from the throat, mouth, or nose of the patient. The practitioner brings the swab 12 into contact with the acquisition port 201 thereby transferring the sample to the acquisition port 201. The card 14 is then sealed and inserted into the device using a slotted entry 301 or other means devised to firmly and properly seat the card 14 into the device 16. The device 16 obtains any pertinent information from bar codes or similar information storage means by using a bar code reader or other well-known means. If necessary, the device 16 generates information that appears in the display 320 indicating that a particular module 500 carrying specific reagents in reservoirs 502, 504, 506 is required to carry out the nucleic acid assays. The correct module 500 is placed into the device 16 and the device 16 is activated using the keypad 345. The device 16 provides electrical and physical communication to the card 14 in order to automatically carry out the assay in a particular order by opening and closing valves on the card 14 in order to bring the appropriate sample, reagents, and physical changes (heating and cooling) to the appropriate place on the card 14. One skilled in the art recognizes various ways to control the valves and pumping action on the card 14. For instance, U.S. Pat. No. 6,767,194 describes micro-fluidic systems including valves and pumps for micro-fluidic systems.

The device 16 provides mechanical energy to drive the fluids to the desired place on the card by using positive air pressure applied to the air ports by the pump 510 or by the on board peristaltic pump 416. After the device 16 has performed the lysing, isolating, washing, amplify and detection steps, the microprocessor 340 analyses the results of the assays and reports the results via the display 320, and/or the printer 325, and/or the communications port 330.

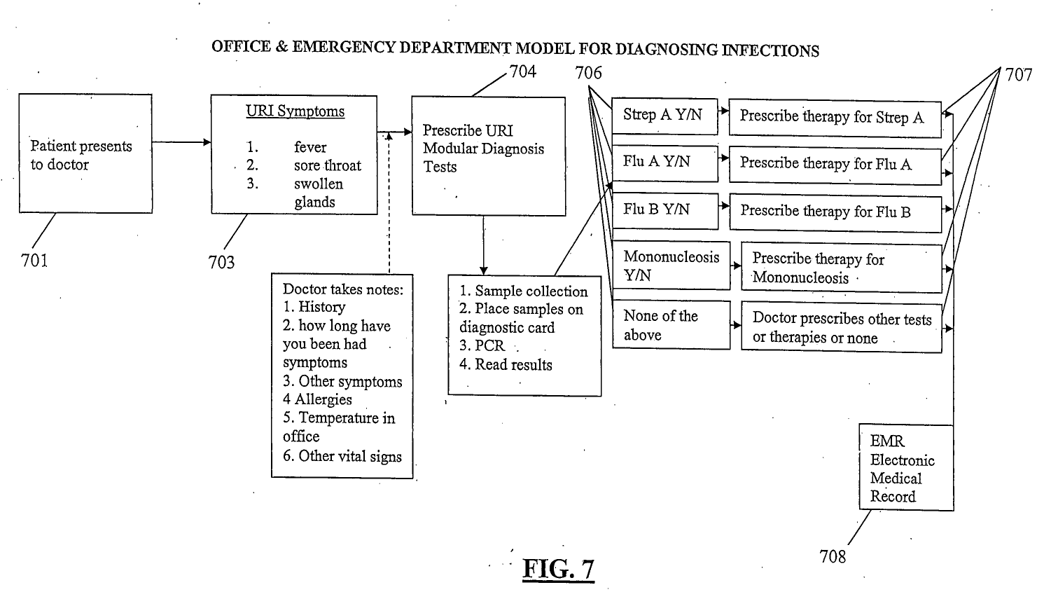

Referring now to FIG. 7, the schematic shows the network and process that is enabled by the rapid diagnostic card. By providing rapid detection services for pathogens, diagnosis of ailments in general can be accomplished without face-to-face contact with medical professions. For instance, a patient can present 701 by way of any form of communication to a physician and certain symptoms can be noted 703 by the physician. The physician can then direct the use of the correct modular diagnosis kit 704 which will verify that a sample has been collected, and that the results have indicated a particular pathogen 706 or pathogens. At that point the correct therapy is prescribed 707 to the particular pathogen and that treatment recommendation can be reported to a means for receiving an electronic medical record 708.

It can be anticipated that this method can be practiced by way of any communication means. So it is possible that verifiable means for recording temperature, blood pressure, and input of other symptoms could be collected by a digital recording means and assembled into a record that could be sent over the internet to a medical professional, that a diagnostic card could be used, its identity noted and the results could also be provided via a network to the medical professional, and in combination the physician could make a diagnosis.

For example, an electronically administered questionnaire could be answered over the internet, such as in a secure form over the internet, and transmitted. As described before, the physician could identify the correct diagnostic card to be used, and remotely the sample and testing of the sample could be accomplished, and the results transmitted in order to provide an improved basis for diagnosis of the patient. This could extend the realm of medical care and oversight beyond normal treatment environments into the field, into homes, remote locations, and emergency conditions.

1. A network for assessing medical conditions comprising:
means for guiding inquiry into a patient's observable symptoms which will provide an affirmative signal if conditions are met to provide a biological sample;
a diagnostic card configured to enable at least one nucleic acid diagnostic assay for rapidly detecting the presence or absence of multiple pathogens at the point-of-care; and
means for instructing said diagnostic card to test said sample to identify the specific pathogens present in said sample.

2. The network of claim 1 wherein the network has further means for adding a patient's vital signs.

3. The network of claim 1 wherein said observable symptoms include fever, swollen glands, and a sore throat.

4. The network of claim 1 wherein said multiple pathogens share a common clinical manifestation.

5. The network of claim 1 wherein said multiple pathogens include at least a first pathogen selected from the group consisting of a virus, a bacterium, a fungus, and a parasite and at least a second pathogen selected from the group consisting of a virus, a bacterium, a fungus, and a parasite.

6. The network of claim 5 wherein the network has further means to provide the identity of said detected pathogens to a physician.

7. A diagnostic system comprising: a diagnostic card configured to enable at least one nucleic acid diagnostic assay for rapidly detecting the presence or absence of multiple pathogens at the point-of-care.

8. The system of claim 7 wherein said multiple pathogens share a common clinical manifestation.

9. The system of claim 7 wherein said multiple pathogens include at least a first pathogen selected from the group consisting of a virus, a bacterium, a fungus, and a parasite and at least a second pathogen selected from the group consisting of a virus, a bacterium, a fungus, and a parasite.

10. The system of claim 7 further comprising a device that communicates with said diagnostic card.

11. The system of claim 10 further comprising analyses means to analyze data from said diagnostic card.

12. The system of claim 11 wherein a treatment regimen is included.

13. The system of claim 11 further comprising communication means to a network to provide test results to a medical practitioner so that said practitioner may provide diagnosis.

14. The system of claim 11 further comprising communication means to a network to provide test results to a medical practitioner so that said practitioner may provide a treatment regimen.

15. A method of diagnosing the underlying cause of a common clinical manifestation, said method comprising: providing a diagnostic card configured to enable at least one nucleic acid diagnostic assay for rapidly detecting multiple pathogens at the point-of-care; introducing at least one sample into said card; interacting said card with a device; and means connected to said diagnostic card wherein said means are capable of instructing said diagnostic card to test said sample to identify the specific pathogens present in said sample.

16. The method of claim 15 wherein said device has analyzing means for determining the results of said diagnostic assay.

17. The method of claim 15 wherein said multiple pathogens share a common clinical manifestation.

18. The method of claim 15 wherein said multiple pathogens include at least a first pathogen selected from the group consisting of a virus, a bacterium, a fungus, and a parasite and at least a second pathogen selected from the group consisting of a virus, a bacterium, a fungus, and a parasite.

19. A method of diagnosing the underlying cause of a common clinical manifestation, said method comprising: providing a diagnostic card configured to enable at least one nucleic acid diagnostic assay for rapidly detecting multiple pathogens at the point-of-care; introducing at least one sample into said card.

20. The method of claim 19 wherein said multiple pathogens share a common clinical manifestation.

21. The method of claim 19 wherein said multiple pathogens include at least a first pathogen selected from the group consisting of a virus, a bacterium, a fungus, and a parasite and at least a second pathogen selected from the group consisting of a virus, a bacterium, a fungus, and a parasite.

22. The method of claim 19 wherein said assays comprise DNA assays.

23. The method of claim 19 wherein said assays comprise RNA assays.

24. The method of claim 19 wherein said nucleic acid assays include an amplification phase.

25. The method of claim 24 wherein said amplification phase comprises polymerase chain reaction.

26. The method of claim 19 wherein said nucleic acid assays include a positive control and a negative control for each of said multiple pathogens.

27. The method of claim 20 wherein said common clinical manifestation comprises at least one of a sore throat, swollen lymph nodes, and fever.

28. The method of claim 27 wherein said assays are designed to detect the presence of at least two of the group consisting of Streptococcus Pyogenes, Influenza A, Influenza B, and Epstein-Barr Virus.

29. The method of claim 19 further comprising a sample port that is sealed after a sample is introduced to said diagnostic card.

30. The method of claim 19 wherein said card utilizes at least one sample to detect the presence or absence of any of said pathogens.

31. The method of claim 30 wherein said at least one sample is introduced into said card by a sample collection device.

32. The method of claim 19 wherein at least one reagent for said assays is contained on board said card.

33. The method of claim 32 wherein said at least one reagent selected from the group consisting of a lysis reagent, an elution reagent, a rehydrating fluid, air, and polymerase chain reaction reagents for said nucleic acid assay is contained on board said card.

34. The method of claim 19 further comprising a chamber configured to allow for rapid thermal cycling for conducting polymerase chain reaction.
